(19) United States
(12) Patent Application Publication
(10) Pub. No.: US 2024/0387058 A1
(43) Pub. Date: Nov. 21, 2024

(54) DIGITAL MEDICINE COMPANION FOR TREATING AND MANAGING SKIN DISEASES

(71) Applicant: PFIZER INC., New York, NY (US)

(72) Inventors: Dennis P. HANCOCK, Collegeville, PA (US); Joshua RAYSMAN, San Diego, CA (US); Felicia ZFIRA, New York, NY (US); Anthony LAMBROU, New York, NY (US); Robert Michael DAY, Walton Oaks (GB); Urs KERKMANN, Berlin (DE); Adam FITZGERALD, Boston, MA (US); Timothy MCCARTHY, Cambridge, MA (US); Carrie Annalice NORTHCOTT, Cambridge, MA (US); Yiorgos CHRISTAKIS, Cambridge, MA (US); Fahimeh MAMASHLI, Cambridge, MA (US); Junrui DI, Cambridge, MA (US)

(21) Appl. No.: 18/722,084

(22) PCT Filed: Dec. 16, 2022
(86) PCT No.: PCT/IB2022/062397

§ 371 Date: Jun. 20, 2024

Related U.S. Application Data
(60) Provisional application No. 63/291,798, filed on Dec. 20, 2021.

(51) Int. Cl. G16H 50/70; G16H 10/60; G16H 20/10; G16H 50/20; G16H 80/00

(52) U.S. Cl. CPC G16H 50/70; G16H 10/60; G16H 20/10; G16H 50/20; G16H 80/00

(57) ABSTRACT

A digital medicine companion for managing skin diseases (e.g., atopic dermatitis, psoriasis) may include patient wearable devices passively collecting patient data, patient user devices with healthcare applications for the patient to enter health related data, central analytics for flare prediction and disease progress tracking, and a clinician dashboard. For flare prediction, a prediction tool may be trained using the ground truth of recorded flare occurrences for the trained model to predict whether observed scratch events may result in a flare. Upon predicting a likely flare, alert notifications may be generated, e.g., for a clinician and/or the patient. Furthermore, a baseline may be established based on the data passively gathered from the wearables and actively gathered from the patient user devices. The continuously collected data may be compared against the established baseline. Upon detecting a significant deviation, alerts may be sent to the patient and/or the clinician.

### RELATED APPLICATIONS

This application claims priority to U.S. Provisional Application No. 63/291,798 filed on Dec. 20, 2021; This application is also related to PCT Application No. PCT/US21/38699 filed Jun. 23, 2021, which claims priority from U.S. Provisional Application No. 63/043,108, filed Jun. 23, 2020 and U.S. Provisional Application No. 63/213,592, filed Jun. 22, 2021; each of which has been incorporated herein by reference in its entirety.

### FIELD

The subject matter presented herein is directed to systems and methods for monitoring skin diseases. Specifically, systems and methods for monitoring scratching caused by skin diseases and the prediction of a likelihood of a flare.

### BACKGROUND

Chronic relapsing and remitting skin conditions/diseases such as atopic dermatitis and psoriasis are characterized by flares. A flare is a significant exacerbation of the disease—involving extreme itchiness, increase in scratching, skin lesions, reductions in sleep, and other associated physical-mental-social discomforts. Managing flares is therefore an integral part of successfully managing these skin diseases.

Managing flares conventionally has been reactive and therefore remains inadequate. A patient may experience a flare and then seek medical attention, generally not available immediately and often not convenient. In the meantime, the flare may—in addition to causing general discomfort of itching and physical action of scratching—produce highly visible skin lesions thereby inviting a form of social stigma. Therefore, as the flare occurs and progresses, the patient's quality of life may significantly decrease. In addition, skin diseases has been found to be associated with psychological problems, depression, anxiety, self-injurious behavior, obesity, food allergies, asthma, and allergic rhinitis/rhino-conjunctivitis; which all may be exacerbated by the flares. A post-flare, reactive medical intervention, having the potential risks of the patient developing and/or aggravating these and other conditions, is therefore inadequate.

As with managing flares, conventional skin disease management is inadequate for managing other associated uncertainties too. As a disease relapses and remits, patients have difficulty monitoring and reporting the progression, given the disease's dynamic nature. The patients' report to the clinicians may be inaccurate and prone to different human biases, thus creating an extra challenge to the clinicians when determining minimally sufficient medication dosing. The uncertainties persist after the medication is prescribed. Patients may drop off the medication if adequate support is not provided to manage side effects (e.g., nausea), even though the medication gains efficacy with continued use. Remission may also cause patients to stop taking medications. Clinicians tend to manage these uncertainties in clinical encounters with the patients; however these encounters however may be a few and far between, influenced by human factors, and may involve inaccurate information exchanges. There may be other associated symptoms such as anxiety, sleeplessness, and fatigue, which cannot be adequately detected and addressed by the clinicians in these clinical encounters and are dependent on a patients' recollections. These symptoms may require a constant intervention to encourage better behavioral choices in exercise, nutrition, mindfulness, etc. The patients may also have to be provided relevant clinical information about the diseases for them to understand and manage the diseases. The usage of these sporadic clinical encounters for disease tracking, determining minimally sufficient dosing, managing side effects, promoting adherence to medication, providing information about the disease, and encouraging better disease management behavior has been inadequate.

### SUMMARY

In some embodiments, the present disclosure relates to a digital medicine companion for treating and managing skin diseases (e.g., atopic dermatitis, psoriasis, etc.) may include patient wearable devices and/or invisible devices (e.g., non-wearable sensors) passively collecting patient movement and/or biological data, patient user devices with healthcare applications for the patient to enter health related data, central analytics for flare prediction and disease progress tracking, and a clinician dashboard. For flare prediction/assessment, a prediction tool may be trained using a supervised training approach using the ground truth of recorded flare occurrences such that the trained model may predict whether the currently observed scratch events and other contextual data may likely result in a flare. In response to a prediction of a likely flare one or more alert notifications may be generated, e.g., for a clinician to increase dosage and/or the patient to take the increased dosage. For managing atopic skin diseases, a baseline may be established based on the data passively gathered from digital technology (e.g., patient wearables and/or invisible devices) and actively gathered from the patient. These acquired data may be compared against the established baselines to evaluate any deviations. In response to detecting a significant deviation in disease presentation for a patient, alerts may be sent to the patient and/or the clinician (in some embodiments, to scientists for further research). Furthermore, a two-way connectivity may be established between the patient and the clinician through the healthcare application and the clinician dashboard, respectively.

In an embodiment, a computer implemented method may be provided. The method may include retrieving, by a server, a scratch dataset, the scratch dataset comprising data records of corresponding scratch events of a patient population with a skin disease; retrieving, by the server, a contextual dataset comprising data records of additional information associated with the corresponding scratch events; training, by the server, a prediction model using a supervised training approach based on the scratch dataset of the contextual dataset; receiving, by the server, periodic data indicating occurrences of scratch events for a time period of a particular patient with the skin disease; feeding, by the server, the received periodic data into the trained prediction model; and in response to the prediction model outputting a likelihood of a flare, transmitting, by the server, an alert notification to a user device of the particular patient.

In another embodiment, another computer implemented method may be presented. The method may include periodically receiving, by a server, healthcare data from a healthcare application installed on a user device of a patient with a skin disease for a predetermined time period; establishing, by the server, a baseline health behavior based on the healthcare data for the predetermined time period; receiving, by the server, new healthcare data from the healthcare application installed on the user device; determining, by the server, whether the new healthcare data has a significant deviation from the baseline health behavior; and in response to the server determining a significant deviation from the baseline health behavior, triggering an alert notification to a clinician dashboard.

In yet another embodiment, yet another computer implemented method may be provided. The method may include continuously receiving, by a computing device, healthcare data of a patient having a skin disease, from a wearable computing device worn by the patient; periodically prompting, by the computing device, the patient to enter additional healthcare data in an interface generated by a healthcare application installed in the computing device; transmitting, by the computing device to a remote server, the healthcare data and additional healthcare data; receiving, by the computing device from the remote server, an alert notification in response to the remote server determining a likelihood of worsening of the skin disease; and in response to receiving the alert notification, generating, by the computing device, a push notification indicating an action for the patient.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIGS. 1A-1F show graphs illustrating different types of disease progression of atopic dermatitis, an example skin disease.

FIG. 2 shows a graph illustrating a prior art clinical assessment of atopic dermatitis.

FIG. 3 shows a graph illustrating a prior art reactive management of atopic dermatitis flares.

FIG. 4 shows a graph illustrating a proactive management of skin disease such as atopic dermatitis, which may be achieved employing one or more embodiments of this disclosure.

FIG. 5 shows a block diagram of an illustrative operating environment for employing one or more embodiments of this disclosure.

FIG. 6 shows a block diagram of an architecture of an illustrative computing device that may perform one or more functions, according to the several embodiments of this disclosure.

FIG. 7 shows a block diagram of an illustrative architecture of operating environment 700, wherein one or more embodiments disclosed herein may be employed.

FIG. 8 shows a flow diagram of an illustrative method of training a prediction model for flare prediction, according to the several embodiments of this disclosure.

FIG. 9 shows a flow diagram of an illustrative method of deploying a prediction model for flare prediction, according to the several embodiments of this disclosure.

FIG. 10 shows a flow diagram of an illustrative method of generating an analytical model of a baseline disease behavior of a skin disease, according to the several embodiments of this disclosure.

FIG. 11 shows a flow diagram of an illustrative method of deploying the analytical model to determine whether the current observed behavior deviates significantly from the baseline disease behavior, according to several embodiments of this disclosure.

FIG. 12A-12D show an illustrative patient facing interfaces, according to several embodiments of this disclosure.

FIGS. 13A-13C show clinician facing interfaces, according to several embodiments of this disclosure.

The figures are for purposes of illustrating example embodiments, but it is understood that the present disclosure is not limited to the arrangements and instrumentality shown in the drawings. In the figures, identical reference numbers identify at least generally similar elements.

### DESCRIPTION

Skin diseases/conditions (e.g., atopic dermatitis, psoriasis, etc.) are characterized by flares and exacerbation over the years. There are also other uncertainties associated with skin disease: the diseases are volatile, they may leave the skin looking clinically normal while being immunologically abnormal, and their long-term management may require the patients adhering to strict treatment regimens and making better behavioral choices. There is also a social stigma owing to the highly visible lesions, formed particularly by the flares. The disease is also associated with psychosocial problems, depression, anxiety, self-injurious behavior, obesity, food allergies, asthma, and allergic rhinitis/rhino-conjunctivitis, etc.

The conventional methods of managing skin diseases remain inadequate. For instance, the flares are generally episodic and the managing these flares has conventionally been reactive. For example, a patient may reach out to a clinician after a flare, and the clinician may prescribe a medication (e.g., a higher dosage of an existing medication and/or a new medication). This reactive management carries the risk of the flare getting even worse and aggravating other conditions such as the psychosocial problems, depression, anxiety, etc. It is therefore desirable to have methods of proactively managing the predicted flares.

As skin diseases are generally dynamic and their progression may ebb and flow. Patients therefore may have difficulty tracking progress and may not be able to recall the full story during clinical encounters, or it may have resolved by the time they are able to interact with their clinician. In other words, the clinicians may not have a complete picture of disease progression during these encounters and therefore may struggle with prescribing a minimally sufficient dosing of a prescription (e.g., Abrocitinib for atopic dermatitis). Furthermore, the prescription may have unpleasant side effects (e.g., nausea) and the patients may drop off treatment if proper counseling is not provided to manage these side effects. Remission may also case the patients to stop taking the medication. Furthermore, without a fuller picture of the patients' reaction to the medication, doctors may be concerned about long term safety of the prescribed medications. Additionally, skin diseases may be associated with other diseases such as anxiety, sleep, and fatigue that may not be addressed by clinicians generally focused on solving the visible problem (i.e., the skin disease itself) during the clinical encounters. These other diseases may have to addressed together with the skin conditions, but these diseases may not be apparent during the few clinical encounters without a continuous monitoring of and an ongoing dialogue with the patients. Therefore, the conventional method of reactive clinical encounters more focused on solving visible acute problems is inadequate on several fronts.

Embodiments disclosed herein attempt to predict and proactively avoid flares of skin diseases. Embodiments disclosed herein may further allow a continuous monitoring of the patients: gathering data about their disease condition and maintaining an ongoing patient-clinician dialogue. To that end, an example operating environment may include devices (e.g., wearable devices, invisible devices) that may passively collect patient data (e.g., without affirmative acts by the patients), such has hand movements and healthcare applications that the patient may be actively prompted to enter disease information (e.g., how they have been feeling at a particular point in time). Data from other sensors (e.g., blood pressure monitors) and contextual data (e.g., EHR data, weather data) may also be received. A back-end prediction model may use the collected data to determine a likelihood of a flare. In some embodiments, the flare prediction may be based on using scratch detection techniques disclosed on PCT Application No. PCT/US21/38699, which has been incorporated herein in its entirety by reference. A prediction of a likely flare may trigger one or more alert notifications: to the patient indicating that a flare is imminent and a remedial action may be taken and/or to the clinician that the particular patient may be prone to an imminent flare. Based on these alerts the patient and the clinician may initiate a synchronous or asynchronous communication and decide on remedial measures accordingly (e.g., increasing the dosage of the prescription medication such as Abrocitinib for atopic dermatitis). The communication between the clinician and the patient may also be used for educational purposes, the clinician may counsel the patient on how to mitigate future flares, how to make healthy behavioral choices on food and exercise, etc.

Alternatively or additionally, the example operating environment may also be used for skin disease progression tracking. For instance, using the patient facing healthcare application, a patient may periodically enter the healthcare data, e.g., side effects, flare status, and/or any other quality of life metric. The wearable (and/or invisible) may also passively capture the patient data, e.g., movement and exercise data. In response, the patient may regularly receive educational materials (e.g., audio, video, text, person to person live counseling) on to how to manage the disease condition and make healthy behavioral choices. The continuously gathered longitudinal data (both passive, active, and/or any other contextual data) may be used to establish a statistical analytic model (and/or train a machine learning model), which may indicate a baseline disease behavior. The baseline disease behavior may indicate normal levels of scratch events, stress, anxiety, and/or any other heath or quality of life metric. New incoming data may be compared against the analytic models to determine whether the new data indicates a significant deviation from the baseline, e.g., whether a side effect is particularly pronounced. In such a case, alert notifications may be generated to the patient and/or the clinician. Based on the alert notification, the patient and the clinician may initiate synchronous or asynchronous communication to decide on remedial measures. The communication may also be used to continuously educate and counsel the patient for better disease management and improve the quality of life.

Therefore, compared to the conventional reactive and sporadic clinical encounters with sub-optimal information exchange, embodiments disclosed herein may allow a continuous and proactive patient monitoring. The back-end analytics may use the continuously gathered data to predict an adverse condition (e.g., an imminent flare) or detect an aggravation (e.g., side effect symptoms getting worse). Such problems may be immediately flagged and addressed through the alert notifications sent to the patients and clinicians. Such monitoring and prediction/detection of worsening of the disease may help clinicians decide on minimally sufficient dosing. Furthermore, a two-way connectivity between the patients and clinicians may allow the clinicians to continuously educate and counsel the patients.

Generally, embodiments disclosed herein may provide more context and understanding the skin diseases thereby allowing for a better treatment. Furthermore, patients may have a sense of “ownership of the disease” as they will be active participants in its treatment. The clinicians may have a fuller picture based on the continuously collected data. Early treatment options may be explored and used based on the collected data. The treatment may be continuously and dynamically modulated (e.g., by adjusting the drug dosage). The safety of the treatment may be increased as heavy and/or continuous dosages may not be needed. Furthermore, the environmental impact on the disease progression may be detected and mitigated.

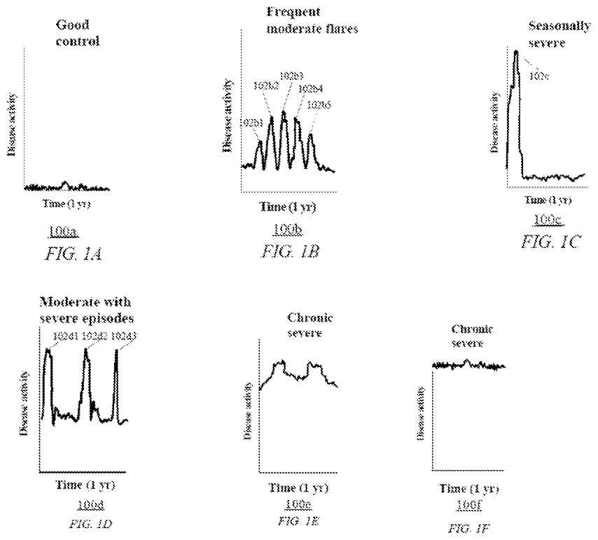

FIGS. 1A-1F show graphs (modified from Jonathan Silverberg, “Atopic Dermatitis: Clinical Features, Patient Type, Burden, and Epidemiology”) illustrating different types of disease progression of atopic dermatitis, an example skin disease. Particularly, FIG. 1A shows a graph 100a illustrating a good control of atopic dermatitis. As shown, disease activity has remained consistently lower over the course of a year and there have been no flares. FIG. 1B shows a graph 100b illustrating atopic dermatitis with frequent moderate flares. As shown, over the course of a year, five moderate flares 102b1, 102b2, 102b3, 102b4, and 102b5 have been observed. FIG. 1C shows a graph 100c illustrating atopic dermatitis with a seasonally severe flare. As shown, a severe flare 102c has been observed at the beginning of the year. FIG. 1D shows a graph 100d illustrating a moderate atopic dermatitis with several severe flare episodes. As shown, although the baseline of atopic dermatitis is moderate, three severe flares 102d1, 102d2, and 102d3 have be observed over the course of the year. FIG. 1E shows a graph 100e illustrating a severe case of atopic dermatitis with a significantly higher disease activity. FIG. 1F shows a graph 100f illustrating another severe case of atopic dermatitis, also with significantly higher disease activity.

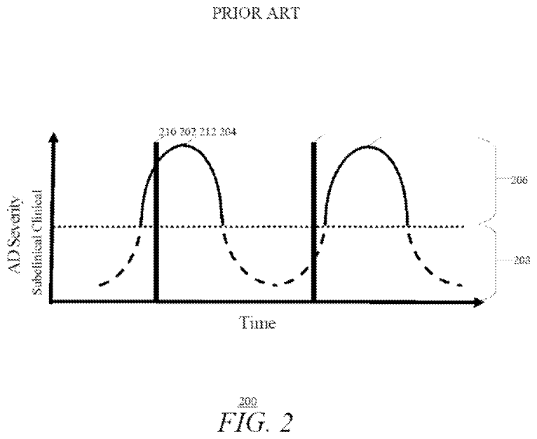

FIG. 2 shows a graph 200 illustrating a prior art clinical assessment of atopic dermatitis. As shown, the atomic dermatitis may fluctuate between subclinical inflammations 208 and clinical inflammations 206. The subclinical inflammations 208 may not generally be observed through clinical tools (e.g., a naked eye observation), and the clinical inflammations 206 may be observed using clinical tools. The clinical inflammations 206 shows two peaks 202 and 204, which may correspond to flares. In the graph 200, two clinical assessment points 210 and 212 have been shown. However, both the clinical assessment points 210 and 212 are at distinct points in time and therefore privy just these point observations. Particularly, the clinical assessment point 210 may detect a likely severe inflammation because of the clinical assessment point 210 is close to the first peak 202. On the other hand, the clinical assessment point 212 may not detect any inflammation as the existing inflammation is a non-observable subclinical inflammation 208. In other words, the clinical assessment points 210 and 212 may not detect long term trends of the disease progression, but just short-term visible conditions. Therefore, current clinical encounters are inherently geared towards detecting and attempting to address atopic dermatitis in a short term and do not provide an optimal long-term management of the disease.

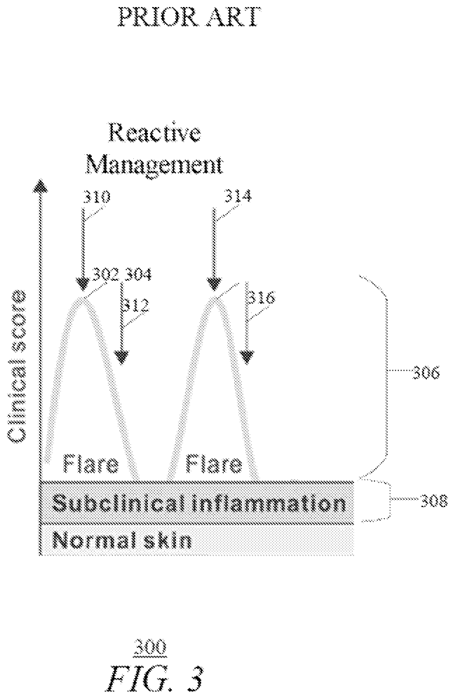

FIG. 3 shows a graph 300 (modified from Thomas Bieber, “Atopic Dermatitis” Ann Dermatol. 2010 May; 22 (2): 125-137) illustrating a prior art reactive management of atopic dermatitis flares. As shown, the atopic dermatitis, which ranges from subclinical inflammation 308 and clinically detectable inflammation 306, has two flares 302 and 304. The clinical interventions 310, 312, 314, and 316 are however just reactive to the flares 302 and 304. Particularly, the patient may have sought clinical help after the flares 302 and 304 were observed. Such reactive management is inadequate at least because the patient has to experience the physical, mental, and social discomforts of having the flares 302 and 304 in the first place. To avoid these and other discomforts, it is desirable to predict and proactively avoid having the flares.

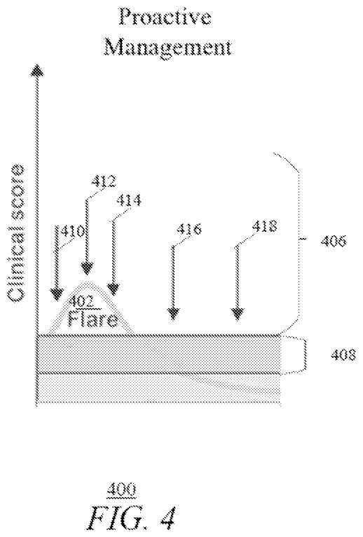

FIG. 4 shows a graph 400 (modified from Thomas Bieber, “Atopic Dermatitis” Ann Dermatol. 2010 May; 22 (2): 125-137) illustrating a proactive management of a skin disease (e.g., atopic dermatitis), which may be achieved employing one or more embodiments of this disclosure. As shown, a first clinical intervention 410 begins before a moderate flare 402. In other words, the flare 402 may have been predicted and the clinical intervention 410 may have reduced the severity of the flare 402. There is another clinical intervention 412 at the peak of the flare 402 for its management. Another clinical intervention 414 is also geared towards managing the flare 402. Subsequent clinical encounters 416 and 418 are geared towards proactively avoiding the flares.

However, the current sporadic, reactive clinical encounters are inadequate for such proactive management of flares. The clinical encounters are inherently geared towards short term cures of subsiding the existing flares. Because clinicians conventionally do not have access to the longitudinal data accumulated over time, they may not be able to optimally manage atopic dermatitis (and generally any other skin disease) in the long run.

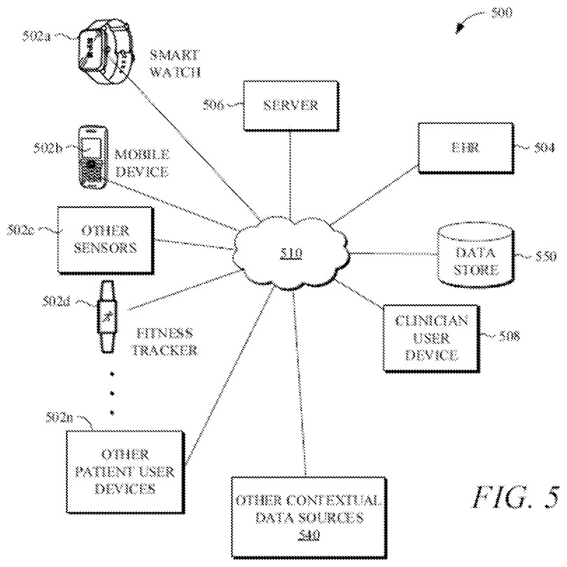

FIG. 5 shows a block diagram of an illustrative operating environment 500 for employing one or more embodiments of this disclosure. It should be understood that this and other arrangements described herein are set forth only as examples. Other arrangements and elements (e.g., machines, interfaces, functions, orders, and groupings of functions) can be used in addition to, or instead of, those shown in FIG. 5 as well as other figures, and some elements may be omitted altogether for the sake of clarity. Further, many of the elements described herein are functional entities that may be implemented as discrete or distributed components or in conjunction with other components, and in any suitable combination and location. Various functions or operations described herein are being performed by one or more entities including a hardware, firmware, software, and a combination thereof. For instance, some functions may be carried out by a processor executing instructions stored in memory.

As shown, the operating environment may comprise patient facing user devices 502a-502n (collectively referred to as devices 502 or commonly referred to as device 502), a server 506, an electronic health record (EHR) system 504, a data store 550, and a clinician user device 508. It should also be understood that the singular or plural description of the devices are just for the sake of clarity in explanation and should not be considered limiting. For instance, the server 506 may include multiple servers and the clinician user device 508 may include multiple user devices. The different devices in the operating environment 500 may be connected to a network 510.

The patient facing user devices 502 may include any type of computing and/or sensing device that the patients may interact with. The non-limiting examples shown in the operating environment 500 may include a smartwatch 502a, a mobile device 502b (e.g., a smartphone), other smart sensors 502c (e.g., a smart ring, invisible sensors such as motion sensors, etc.), a fitness tracker 502d, and other patient facing user devices 502n (e.g., tablets, laptop computers, desktop computers, smart speakers, smart home systems, etc.). The patient facing user devices 502 may either passively collect or actively prompt a patient to enter data associated with skin diseases (e.g., atopic dermatitis, psoriasis) for flare prediction and/or for a long-term management of the disease.

For example, the smartwatch 502a may have multiple sensors to passively collect data from the patient. The multiple sensors may include accelerometers, gyroscopes, and/or other types of motion sensors that may track the hand movements of the patient. The hand movements may be used by the operating environment (e.g., one or more software modules in the server 506) to detect scratch events (e.g., by using the embodiments disclosed on the PCT Application No. PCT/US21/38699). In addition to detecting hand movement, the smartwatch 502a passively detect other biological parameters such as blood glucose, body temperature, blood oxygen saturation level, and/or any other type of biological parameter.

The mobile device 502b may be used by the patient for actively entering health related data. For example, the mobile device 502d may be a smartphone that may have a healthcare application installed therein. The healthcare application may prompt the patient to enter health related data. For instance, the healthcare application may generate a push notification for the patient to enter data as to how the patient is feeling at the given point in time. The prompt may be, “How are you feeling this morning?” and the patient may enter, “I am feeling great.” The healthcare application may also display alert notification, which may be generated due to the operating environment predicting that a flare is likely. Another type of alert notification displayed by the healthcare application may be when the current disease behavior of the patient deviates significantly from the established baseline behavior. The healthcare application may further allow the patient to communicate, synchronously or asynchronously, with the clinician.

The other sensors 502c may include devices such as smart rings, skin patches, ingestible sensors, and/or any other type of body attached or non-body attached sensors (generally referred to as invisibles or invisible devices/sensors). The other sensors 502c may detect biological or non-biological data. For instance, other sensors 502c may include a smart fabric measuring a body temperature of the patient. As another example, the other sensors 502c may include a smart home sensor measuring home temperature and/or humidity. As yet another example, the other sensors 502c may include motion sensor that may detect/measure movement within a room. The other patient devices 502n may include any other type of device associated with the patients. For instance, the other patient devices 502n may include tablet computers, laptop computers, desktop computers, and/or any other computing devices associated with the patients and connected to the network 510. Other contextual data sources 540 may include devices providing other contextual data that may provide additional information on the data collected from the patient facing devices 502. For example, the other contextual data sources 540 may provide weather data that may be associated with the data collected from the patient facing devices 502.

The network 510 may include any kind of communication network. For instance, the network 510 may include packet switching network supporting protocols such as TCP/IP. The network 510 may also include circuit switching networks supporting both wired and wireless telephony. The network 510 therefore may include components such as wires, wireless transmitters, wireless receivers, signal repeaters, signal amplifiers, switches, routers, communication satellites, and/or any other type of network and communication devices. Some non-limiting examples of the network 510 may include local area network (LAN), metropolitan area network (MAN), wide area network (WAN) such as the Internet, etc. These are just but a few examples, and any kind of communication linkage between the different components of the operating environment are to be considered within the scope of this disclosure.

The server 506 may include any type of computing devices that may provide the analytical functionality of training and deploying one or machine learning models and/or establishing and deploying statistical analytic models. For instance, the server 506 may train a prediction model using the ground truth, as shown by scratch data and other contextual data, and then use the prediction model to predict a likelihood of a flare when a new scratch and/or contextual data is received. The server 506 may also establish an analytical model based on the continuously collected longitudinal healthcare data, wherein the analytical model may indicate a baseline healthcare behavior. When new healthcare data is received, the server 506 may compare the received data with the analytical model (e.g., against the baseline healthcare behavior) to determine whether the new healthcare data shows a significant deviation from the baseline health behavior. The server 506 may also generate one or more alert notifications, e.g., to patients and/or clinicians, indicating a flare prediction or that the patient's health behavior has significantly deviated from the baseline behavior.

The electronic health record (EHR) 504 may store the health records of the patients. The health records may include, for example, the patients' ongoing condition (e.g., atopic dermatitis), prescribed medications (e.g., Abrocitinib), summaries of clinical encounters, and/or any other healthcare related data associated with the patients. In some embodiments, the EHR 504 may be maintained by healthcare providing entity (e.g., a hospital system).

The data store 550 may include any kind of database storing data collected from various sources within the operating environment 500. For instance, the data store 550 may store data collected, both passively and actively, from the patient facing devices 502. The data store 550 may also store data collected from other contextual data sources 540 (e.g., weather data). Additionally, the data store 550 may store data sourced from EHR 504. Therefore the data source 550 should be understood to store any kind of data in the operating environment 500.

The clinician user device 508 may be any kind of computing device showing a clinician dashboard. Non-limiting examples of the clinician user device may include a mobile phone (e.g., a smartphone), a tablet computer, a laptop computer, a desktop computer, and/or any other type of computing device. The clinician dashboard may show information (e.g., demographic information, location information) and/or one or more alerts associated with the various patients.

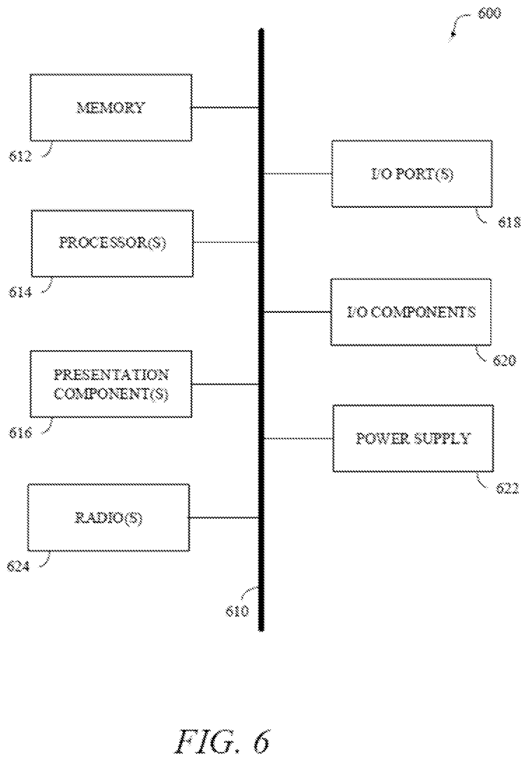

FIG. 6 shows a block diagram of an illustrative computing device 600 that may perform one or more functions described herein, according to the several embodiments of this disclosure. The computing device 600 is but one example of a suitable computing environment and is not intended to suggest any limitation as to the scope of use or functionality of embodiments of the disclosure. Neither should the computing device 600 be interpreted as having any dependency or requirement relating to any one or combination of components illustrated.

Embodiments of the disclosure may be described in the general context of computer code or machine-useable instructions, including computer-useable or computer-executable instructions, such as program modules, being executed by a computer or other machine, such as a personal data assistant, a smartphone, a tablet PC, or other handheld or wearable device, such as a smartwatch. Generally, program modules, including routines, programs, objects, components, data structures, and the like, refer to code that may perform particular tasks or implement particular data types. Embodiments of the disclosure may be practiced in a variety of system configurations, including handheld devices, consumer electronics, general-purpose computers, or more specialty computing devices. Embodiments of the disclosure may also be practiced in distributed computing environments where tasks are performed by remote-processing devices that are linked through a communications network. In a distributed computing environment, program modules may be located in both local and remote computer storage media including memory storage devices.

The computing device 600 may include a bus 610 that may directly or indirectly couple the following example devices: memory 612, one or more processors 614, one or more presentation components 616, one or more input/output (I/O) ports 618, one or more I/O components 620, and a power supply 622. Some embodiments of computing device 600 may further include one or more radios 624. Bus 610 represents what may be one or more busses (such as an address bus, data bus, or combination thereof). Although the various blocks of FIG. 6 are shown with lines for the sake of clarity, these blocks may represent logical, and not necessarily actual, components. For example, one may consider a presentation component such as a display device to be an I/O component. Also, processors 614 may have their memories. Furthermore, distinction is not made between such categories as “workstation,” “server,” “laptop,” or “handheld device,” as all are contemplated within the scope of FIG. 6 and with reference to “computing device.”

The computing device 600 may include a variety of computer-readable media. Computer-readable media can be any available media that can be accessed by computing device 600 and may include both volatile and nonvolatile, removable and non-removable media. By way of example, and not limitation, computer-readable media may comprise computer storage media and communication media. Computer storage media may include both volatile and nonvolatile, removable and non-removable media implemented in any method or technology for storage of information such as computer-readable instructions, data structures, program modules, or other data. Some non-limiting examples of computer readable media may include RAM, ROM, EEPROM, flash memory or other memory technology, CD-ROM, digital versatile disks (DVDs) or other optical disk storage, magnetic cassettes, magnetic tape, magnetic disk storage or other magnetic storage devices, or any other medium which can be used to store the desired information and which can be accessed by computing device 600. Communication media may embody computer-readable instructions, data structures, program modules, or other data in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, communication media may include wired media, such as a wired network or direct-wired connection, and wireless media, such as acoustic, RF, infrared, and other wireless media. Combinations of any of the above should also be included within the scope of computer-readable media.

The memory 612 may include computer storage media in the form of volatile and/or nonvolatile memory. The memory 612 may be removable, non-removable, or a combination thereof. Some non-limiting examples of hardware devices for the memory 1212 include solid-state memory, hard drives, optical-disc drives, etc.

The computing device 600 may include one or more processors 614 that read data from various entities such as memory 612 or the I/O components 620. The presentation component(s) 616 may present data indications to a user or other device. Exemplary presentation components may include a display device, speaker, printing component, and the like.

The I/O ports 618 may allow computing device 600 to be logically coupled to other devices, including I/O components 620, some of which may be built in. Non-limiting examples of the I/O components 620 may include a microphone, joystick, game pad, satellite dish, scanner, printer, or a wireless device. The I/O components 620 may provide a natural user interface (NUI) that processes air gestures, voice, or other physiological inputs generated by a user. In some instances, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye tracking, and touch recognition associated with displays on the computing device 600. The computing device 600 may be equipped with cameras, such as stereoscopic camera systems, infrared camera systems, RGB camera systems, and combinations of these, for gesture detection and recognition. Additionally, the computing device 600 may be equipped with accelerometers or gyroscopes that enable detection of motion.

Some embodiments of computing device 600 may include one or more radio(s) 624 (or similar wireless communication components). The radio may transmit and receive radio or wireless communications. The computing device 600 may be a wireless terminal adapted to receive communications and media over various wireless networks. Computing device 600 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection.

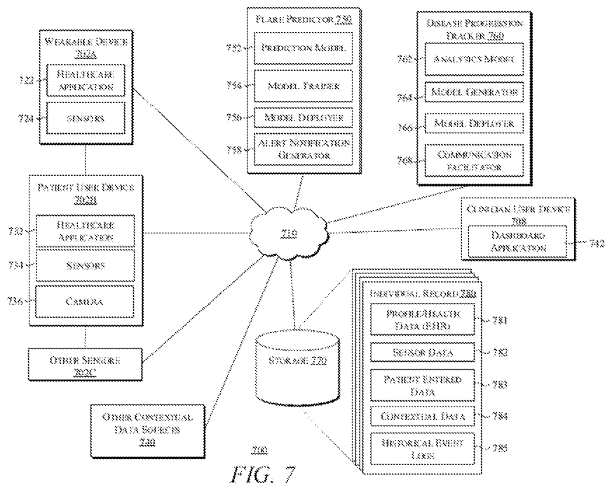

FIG. 7 shows a block diagram of an illustrative architecture of operating environment 700, wherein one or more embodiments disclosed herein may be employed. The shown architecture may be implemented by one of more components/devices of the operating environment 500 shown in FIG. 5. It should be understood that the shown architecture is just but an example and architectures with additional, alternative, or fewer number of components should also be considered within the scope of this disclosure. It should further be understood that components shown as a single components or plural components are also just examples: a single component may include multiple iterations of the same component or multiple constituent sub-components and the functionality of plural components may be achieved by a single component.

The operating environment 700 may be used for a continuous or near-continuous digital monitoring of patients with skin diseases (e.g., atopic dermatitis, psoriasis) and triggering alert notifications to the patients as needed, e.g., when the condition is predicted to worsen or detected to be significantly varying from an established baseline. To that end, the operating environment 700 may comprise patient facing devices (e.g., wearable device 702a, patient user device 702b, other sensors 702c, etc.) to gather healthcare data and other data from the patients, to provide the alert notification to the patients, and to facilitate communication between clinicians and patients. The data gathered from the patient facing devices and other sources (e.g., other contextual data sources 740) may be stored in the storage 770 (e.g., as individual records 780). The analysis components (e.g., flare predictor 780, disease progression tracker 760) may use the stored data and other data to predict flares and to track the progression of skin diseases (e.g., atopic dermatitis, psoriasis). Based on the analysis, alert notifications may be sent to clinicians (e.g., through a clinician user device 708). The components of the operating environment 700 may be interconnected through a network 710.

With regard to the patient facing devices, these devices may include, for example, the wearable device 702a, the patient user device 702b, and other sensors 702c. The wearable device 702a may include any kind of wearable device; non-limiting examples include a smartwatch, a fitness tracker, a smart ring, etc. In some embodiments, the wearable device 702a may include a healthcare application 722. The healthcare application 722 may a computer program installed on the wearable device 702a to collect healthcare data, perform pre-processing of the collected data in some instances, and transmit the data to the patient user device 702b or to a remote server (e.g., implementing one or more of the flare predictor 750, disease progression tracker 760, or storage 770). Particularly, the healthcare application 722 may interface with the operating system of the wearable device (e.g., through API calls) to gather the data from the sensors 724.

The sensors 724 may include any type of sensors that may continuously or periodically gather data from the patient wearing the wearable device 702a. For example, the sensors 724 may include accelerometers to determine directional movement, gyroscopes to detect the orientation, and/or any other type of sensors gather positional or movement data. The sensors 724 may include biological sensors, such as a temperature sensor to measure the body temperature (it should be understood that the temperature sensor may be non-biological and may measure the ambient temperature), heart rate monitor, an electrocardiogram sensor to collect electrocardiogram data when prompted by the patient, a glucose monitor, a sweat monitor, blood oxygen saturation level monitor, and/or any other type biological sensors. These sensors may be triggered by the healthcare application 722 (e.g., through API calls to the operating system of the wearable device 702a) to collect the corresponding data. Alternatively, the wearable device 702a may not have the healthcare application 722 and the triggering may be received from the patient user device 702b (e.g., from its healthcare application 732) or remotely through the network 710. In other embodiments, the wearable device 702a may itself be continuously or periodically activating the sensors 724 and may pass the collected sensor data along to the healthcare application 724 (and/or healthcare application 732 or a remote device connected via the network 710).

In other words, the sensors 724 may be collecting patient data passively, without an active involvement of the patient. For instance, the sensors 724 may be monitoring the patient's physical movements and/or other biological data because the sensors 724 are within the wearable device 702a and are continuously attached to the patient. This passive collection of movement and biological data does not require the patient's continuous attention of the patient and is less burdensome.

The patient user device 702b may include any kind of computing device used by the patient. For example, the patient user device 702b may include a mobile phone such as a smartphone, a tablet device, a laptop computer, a desktop computer, and/or any other type of computing device. A healthcare application 732 may be installed on the patient user device 702b. The healthcare application 732 should be understood to include a standalone application (e.g., a smartphone app) or a web-based application (e.g., accessed using a browser). The healthcare application 732 may provide an interface (e.g., a graphical user interface) for the patient to view alert notifications, communicate with a clinician, and/or actively enter health related data.

As an example, the healthcare application 732 may be used gather further information on a prediction based on the data collected by the sensors 724 of the wearable device. For instance, using the passively collected movement data, a server may determine that the patient a higher than usual scratch events (e.g., as detected by the scratch detection algorithm disclosed in PCT Application No. PCT/US21/38699). In response to this determination, the server may transmit an alert notification to the healthcare application 732. The alert notification may be, for example, “We detected a higher volume of scratch activity last night. How do you feel this morning?” and prompt the patient to respond. For the response, the healthcare application 732 may provide choices such as “Feeling Fatigued,” “Feeling Feverish,” or “Feeling Normal.”

In addition to the prompts corresponding to alert notifications, the healthcare application 732 may request the user to periodically (e.g., without a trigger for data entry) enter healthcare data. For instance, the healthcare application 732 may prompt the patient to enter how they feel every morning—regardless of the overnight scratch activity. Other non-limiting examples of the actively entered data include body temperature (if not collected by the sensors 724 of the wearable device 702a), bowel movement, level of pain experienced, stress level, anxiety level, the time of intake of a prescription medication, exercise activity (if not captured by the wearable device 702a), side effects of the prescription medication, and/or any other type of healthcare data that may affect the quality of life of the patient.

The healthcare application 732 may also provide educational materials to the patient. The educational material in some embodiments may include cognitive behavior therapy (CBT) based behavior modification encouragement materials. These materials may be provided to the patient based on the data passively collected by the wearable device 702a, actively collected by the patient user device 702b, and analyzed by the disease progression tracker 760. The CBT-based materials may be in the form of audio, video, and/or text and encourage the patient to make healthier choices on food, exercise, stress management, and/or any other metric associated with maintaining a good quality of life. The educational material may also be a two-way communication with the clinician. For instance, the healthcare application 732 may provide a communication platform for voice call, video call, and/or an interchange of text messages. The patient may pose questions through the healthcare application 732 itself and the clinician's response may be displayed within the healthcare application. The clinician's response may be educational, advising and encouraging the patient to make healthy choices in terms of managing the skin diseases (e.g., atopic dermatitis, psoriasis).

The sensors 734 in the patient user device 702b may include any type of sensors such as accelerometers, gyroscopes, glucose monitor (e.g., using an infrared camera), etc. In case of the patient user device 702b being a mobile device (e.g., a smartphone), the patient user device 702b too may monitor the user's movement using the sensors 734. The sensors 734 may detect the number of steps taken by the patient throughout the day and/or other activities (e.g., exercise) performed by the user throughout the day. In other words, the sensors 734 too may passively collect movement data of the patient. In some embodiments, the sensors 734 may enable an active data collection. For instance, the sensors 734 may comprise an infrared camera and the patient user device 702b may prompt the patient to hold their finger against the camera to detect biological attributes such as blood glucose level, blood oxygen saturation level, etc.

The camera 736 may include an optical camera that the patient may use to take pictures of the areas affected by the skin disease (e.g., atopic dermatitis, psoriasis). The pictures may be sent to the storage 770 and/or provided to the clinician. The clinician may use the pictures for diagnostic purposes (e.g., to determine whether the condition is improving or worsening) and/or for therapeutic purposes (e.g., to determine whether to adjust the dosage the medication the patient is taking.)

The other sensors 702c may be any kind of sensors measuring one or more physical or biological attributes of the patient. An example of the other sensors 702c may be an ingestible sensor that may be measure the effect on gut activity of a prescription medication. Another example may be a patch sensor that may be attached to the skin to measure attributes such skin temperature and/or the movement of the body part that the patch sensor is attached to. The sensors 702c may further include a blood pressure monitor that may communicate measurements to the patient user device 702b or any other device within the operating environment 700. Other examples of the sensors 702c may include smart fabrics, smart belts, sub-cutaneous sensors, etc. These are just but a few examples of the sensors and any type of body-worn or non-body-worn sensor should be considered within the scope of this disclosure. The non-body worn sensors may be referred to as invisibles or invisible sensors devices.

Other contextual data sources 740 may provide additional context to the data captured by the wearable device 702a, patient user device 702b, and/or other sensors 702c. For instance, the data sources 740 may be weather related data source, providing a general weather condition of the area of where the patient is located. In another instance, the data sources 740 may provide other attributes of the geographical location of the patient, such as prevalence of diseases, general education level, general income level, and/or any other type of contextual data.

The data collected, either passively or actively, by one or more of the wearable device 702a, patient user device 702c, other sensors 702c, and other contextual data sources 740 may be received by other components in the operating environment through the network 710. The network 710 may include any kind of communication network. For instance, the network 710 may include packet switching network supporting protocols such as TCP/IP. The network 710 may also include circuit switching networks supporting both wired and wireless telephony. The network 710 therefore may include components such as wires, wireless transmitters, wireless receivers, signal repeaters, signal amplifiers, switches, routers, communication satellites, and/or any other type of network and communication devices. Some non-limiting examples of the network 710 may include local area network (LAN), metropolitan area network (MAN), wide area network (WAN) such as the Internet, etc. These are just but a few examples, and any kind of communication linkage between the different components of the operating environment are to be considered within the scope of this disclosure.

The data received from the patient facing devices may be stored in the storage 770. The storage 770 may include any kind of storage technology such as hard drive storage, solid state storage, data server storage, etc. Although a single storage 770 is shown for the clarity of explanation, the storage 770 should be understood to include multiple, geographically distributed components. For example, the storage 770 may be distributed among multiple data centers and incorporate multiple level of redundancies.

In some embodiments, the storage 770 may store individual records 780 containing the data for the corresponding patients. In other words, an individual record 780 may be associated with the patient. It should however be understood that this individual record 780 based organization of data is just an example and should not be considered limiting. Any kind of data organization (e.g., relational, object oriented) should be considered within the scope of this disclosure.

As shown an individual record 780 of a patient may include a profile/health data (e.g., electronic health record (EHR) data) 781, sensor data 782, patient entered data 783, contextual data 784, and historical event 785. These are just some examples of the pieces of data within the individual record 780 and additional, alternative, or fewer pieces of data should also be considered within the scope of this disclosure.

The profile/health data 781 may include the electronic health record of the corresponding patient. The profile/health data 781 may therefore include the ongoing disease conditions (e.g., atopic dermatitis, psoriasis) of the patient, medication that the patient is currently taking, allergies, family medical history, clinician encounter summaries, other notes from the clinicians, and/or any other type of healthcare data of the patient. For example, the profile/healthcare data 781 may include information associated with flares—and the information include the timing and severity of the flares. In some embodiments, the profile/health data 781 may be sourced to the storage 770 from other entities. For instance, the profile/health data 781 may be managed by a healthcare providing entity (e.g., a hospital), and the operating environment 700 may retrieve the data from the healthcare providing entity.

The sensor data 781 may be the data from the patient facing sensors such as the sensors 724 of the wearable device 702a, sensors 734 of the patient user device 702b, and/or other sensors 702c. The sensor data 781 may therefore include data from the movement sensors (e.g., accelerometers and/or gyroscopes), biological sensors (e.g., blood glucose monitors). The sensor data 781 may be stored in association with the timestamps of when the data was collected. The timestamps may allow the operating environment 100 to detect the patient's activity throughout the day. As used herein, the sensor data 782 should be understood to include any kind of passively collected data (e.g., movement passively detected by a wearable), or data captured by the patient actively engaging with the sensor (e.g. the patient putting their finger on an infrared camera to measure various biological attributes).

The patient entered data 783 may include any kind of data actively entered by the patient (e.g., through the healthcare application 732). The patient entered data 783 may therefore include, the patient's entry as to how they have been feeling (e.g., “Fatigued,” “Depressed,” “Fine,” etc.) at a particular point in time. The patient entered data 783 may further include other biological data not captured by the sensors (e.g., sensors 724, 734, and/or 702c). For instance, such biological data may include blood glucose level, blood oxygen saturation level, blood pressure, etc. As with the sensor data 782, the patient entered data 783 may also be organized using the timestamps. In other words, the timestamps may be used to correlate the sensor data 782 and the patient entered data 783.

The contextual data 784 may include any kind of information that may provide more context to the sensor data 782 and/or the patient entered data 783. For example, the contextual data may be weather data, which may indicate the weather condition when the corresponding sensor data 782 and/or the patient entered data 783 was collected. As another example, the contextual data 784 may include prevalence of diseases (e.g., atopic dermatitis, psoriasis) in the patient's geographic location, education and income level in the patient's geographic location, and/or any other type of contextual data. As with the sensor data 782 and the patient entered data 783, the contextual data 784 may also be timestamped, such that these three types of data may be temporally correlated.

The historical event logs 785 may include a record of events associated with the patient. For instance, the historical event logs 785 may include scratches detected using the scratch detection algorithm disclosed in PCT Application No. PCT/US21/38699. As another examples, the historical event logs may also include flares as reported by the patient. The historical event logs 785 may include other information on clinical encounters, prescription filling and refilling, and/or any other types of events associated with managing skin diseases (e.g., atopic dermatitis, psoriasis). The historic event logs 785 may also be timestamped such that these logs can be temporally correlated with one or more of the sensor data 782, patient entered data 783, or contextual data 784.

The analytic components (e.g., flare predictor 750 and disease progression tracker 760) may use the individual records 780 in the storage 770 (and/or other types of data) to generate/train one or more analytics/machine learning models, and then deploy the models for one or more of flare prediction or long term management of the disease.

The flare predictor 750 may predict flares based on scratch events (e.g., as detected by the algorithm disclosed in PCT Application No. PCT/US21/38699), contextual data, and/or any other type of data. The flare predictor 750 may use a prediction model 752 to predict the flares. The prediction model 752 may first be trained using a model trainer 752. The module trainer may be include computer program instructions that may retrieve the training data, pre-process the training data, and use the training data to train the prediction model 752 using one or more of supervised or unsupervised training approaches.

In an example training using a supervised training approach, the model trainer may retrieve scratch data (e.g., from historical event logs 785), flare data (e.g., from one or more of the profile/health data 781, patient entered data, contextual data 784, or historic event logs 785), and/or other contextual data (e.g., from the contextual data 784). The flare data may therefore provide a labeling (e.g., “flare” or “no flare”) on the scratch data and/or other input data, thereby allowing the model trainer 754 to minimize the prediction error of the prediction model 752 (e.g., through back propagation). However, backpropagation is just an example and any type of machine learning training should be considered within the scope of this disclosure. Furthermore, the model trainer 754 may train the prediction model 752 using an unsupervised approach. In the prediction model 752 training and deployment embodiments, any type of information associated with detected scratch events may be referred to as contextual data. For instance, the entire individual record 780 or any combination of the constituent data can be referred to as contextual data.

Although the prediction model 752 is described herein as a machine learning model, it should be understood that the prediction model 752 may include a statistical model. For the statistical model, the model trainer 754 may function as a model generator to generate the statistical model. The statistical model may be used for predicting which combinations of input variables (e.g., scratch data and other data) may more likely result in a “flare” output and which combinations of the input variables may more likely result in a “no-flare” output.

It should be understood that the model trainer 754 may continuously train the prediction model 752. For instance, if the ground truth is available for a prediction (e.g., the ground truth may indicate whether the prediction was correct or incorrect), the model trainer 754 may use such correct or incorrect prediction to continuously train and improve upon the prediction model 752.

The model deployer 756 may be software module using the trained prediction model 752 to predict flares from received input data. For example, a new scratch data and/or other contextual data may be received for a particular patient; and the model deployer 756 may feed the received new data into the trained prediction model 752. The trained prediction model 752 may then output the likelihood of a flare based on the input data.

The alert notification generator 758 may generate one or more alert notifications based on the trained prediction model 752 indicating a higher likelihood of a flare based on the received input data. An alert notification may be to the patient user device 702b to be displayed by the healthcare application 732. This patient alert notification may include a message for the patient that the patient should seek medical attention (e.g., communicate with a clinician through the healthcare application 732) to stave off the potential flare. Another example message for the patient may be increase the dosage of the prescription medication (i.e., within the prescribed limits). Overall, the alert notification may trigger the patient to take an action to mitigate the likelihood of the flare.

Another example of the alert notification may an alert notification to a clinician, e.g., to a dashboard application 742 in the clinician user device 708. This alert notification may indicate to the clinician that the patient may have a higher likelihood of the flare. In response to the alert notification, the clinician may communicate with the patient (e.g., using the communication channel provided by the dashboard application 742 and the healthcare application 732) and/or take other actions such as prescribing a higher dosage of the medication.

The disease progression tracker 760 may be another aspect of analytics provided within the operating environment 760. The disease progression tracker 760 may continuously track different aspects of the patient health to determine how the skin disease (e.g., atopic dermatitis, psoriasis) has been progressing, the effect disease on the overall health of the patient, the effect of medication on the disease, and/or any other aspect of the progression of the skin disease. Additionally or alternatively, the disease progression tracker 760 may determine whether the disease condition has significantly deviated from a normal course.

To track the progression of the disease and/or to determine whether the disease condition has deviated significantly from the normal course, the disease progression tracker 760 may generate an analytics model 762 using a model generator 764. The model generator 764 may include computer program instructions that may retrieve long term data from the storage (e.g., profile health data 781, historical event logs 785, etc.). This long-term data may be used by the model generator 764 to generate the analytics model 762. It should however be understood that the analytics model 762 may be a machine learning model and the model generator 764 may use the retrieved data to train the machine learning model. Therefore, the description below of generating and deploying the analytics model 762 should be understood to include training and deploying any type of machine learning model.

In some embodiments, the model generator 764 may generate models for individual patients. For instance, the model generator 764 may retrieve long-term data for individual patients and then establish a baseline as a corresponding analytics model 762. The baseline may include, for example, normal levels of scratch, physical activity, feelings (e.g., “fatigued”) as reported by the patient, and/or any other attribute. The combination of the normal level of these attributes may therefore be established as a baseline.

In some embodiments, the model generator 764 may generate population level baselines. For instance, the model generator 764 may retrieve long-term data for a population of patients with certain criteria, e.g., age, gender, geographical location, ethnicity, etc. Analyzing the collected data, the model generator 764 may establish a population level baseline as the analytics model 762. The model deployer 766 may later use the population level baseline to determine whether an individual patient's condition has deviated significantly from the normal level.

The communication facilitator 768 may facilitate a communication between the patient and the patient, e.g., by using the healthcare application 732 and the dashboard application 742. For example, of the analytics model 762 determines that the state of the diseases has significantly deviated from normal, the communication facilitator 768 may transmit a first alert notification to the patient (e.g., to be displayed on the healthcare application) and a second alert notification to the clinician (e.g., to be displayed on the dashboard application 742). One or more of these alerts may have a communication prompt. For instance, the first alert to the patient may have a prompt “Send A Message To My Doctor.” The second alert to the clinician may be “Reach Out to Patient A, His Dermatitis May Be Getting Worse.” In response to these prompts, an asynchronous (e.g., through text message exchange) or synchronous (e.g., audio/video chat) communication channel may be opened between the healthcare application 732 and the dashboard application 742.

The clinician user device 708 may be any kind of user device used by a clinician. Non-limiting examples of the clinician user device 708 may include mobile phone (e.g., smartphone), tablet computer, laptop computer, desktop computer, etc. The clinician user device 708 may have a dashboard application 742, which may be an installed stand-alone application or web-based application accessible through a browser. The dashboard application 742 may show the disease progress of an individual patient. For instance, the dashboard application 742 may show a chart showing how the scratch events have increased or decreased over time. The dashboard application may also other aspects of the patient's health, e.g., levels of stress and anxiety, etc. The dashboard application 742 may further show the medications prescribed for the patient.

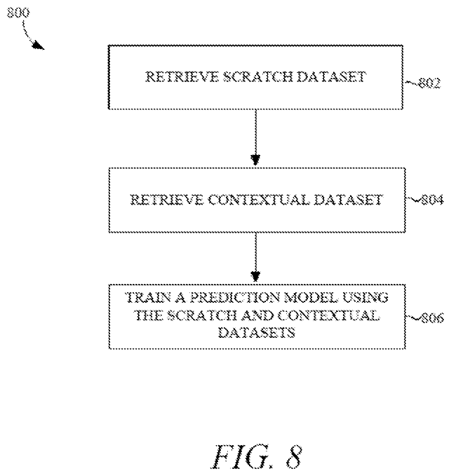

FIG. 8 shows a flow diagram of an illustrative method 800 of training a prediction model for flare prediction, according to some embodiments of this disclosure. The illustrative method 800 may be implemented by one or more computing devices (e.g., computing device 600 as used in the operating environment 500). It should be understood that the steps of the method 800 shown in FIG. 8 and described herein are merely illustrative and methods with additional, alternative, or a fewer number of steps should be considered within the scope of this disclosure.

At step 802, a scratch dataset may be retrieved. The scratch dataset may be generated by the embodiments disclosed in the PCT App. No. PCT/US21/38699. The scratch dataset may include data for a population of patients. For each patient, the corresponding data may include number of scratches (or scratch events) over the course of a time. For example, the number of scratches may be organized on an hourly basis, daily basis, weekly basis, and/or any other time unit of organization. For the scratch events, the corresponding data may also comprise the duration of a scratch. As an example, if a certain patient has three scratch events over the course of an hour, the first scratch event may be for three minutes, the second scratch event may be for five minutes, and the third scratch event may be for two minutes.

The scratch dataset may further include data indicating the severity of scratches. In some embodiments, the duration of a scratch event may serve as a proxy for the severity of the scratch. For instance, a longer scratch event may be deemed more severe than a shorter scratch event. In other embodiments, the severity may be measured by the how vigorous the scratches were for an individual scratch event. In other words, a scratch event with a higher number of hand movements may be more severe compared to another scratch event with a lower number of hand movements. Additionally, the forcefulness of the hand movements may also be recorded, e.g., using the accelerometer data. For instance, a faster hand movement may indicate a more forceful scratching compared to a slower hand movement. These are just but a few examples of the data in the scratch dataset and other form of scratch data should also be considered within the scope of this disclosure.

At step 804, a contextual dataset may be received. The contextual dataset may be received from various sources such as EHR (e.g., EHR 781 shown in FIG. 7), weather forecast data, prescription data, and/or any other type of patient data. The contextual dataset may be associated with the scratch dataset and may provide additional information, e.g., whether the patient experienced a flare.

In some embodiments, the contextual data may provide whether the patient experienced a flare. For example, the EHR may indicate that a flare was detected and/or medication was prescribed for the flare. Alternatively, the patient may have entered in a healthcare application that a flare was experienced, which may be captured as a contextual data. As described with respect to FIG. 7, the contextual dataset may be from various sources and provide an indication whether the patient experienced a flare.

In addition to indicating whether the patient experienced a flare, the contextual dataset may include additional information associated with the scratch events. The additional information may include, for a scratch event, the demographic information of the patient, the weather pattern at the time the scratch event was detected, the geographical location of the patient, prescription medication taken by the patient, the patient's health condition (e.g., diabetes, high blood pressure, psychological diseases), family history of the patient, etc.

Therefore, the combination of scratch dataset and the contextual dataset may provide a labeled dataset of training a prediction model in step 806. Particularly, the scratch dataset and some aspects of the contextual dataset may provide input parameters for training the prediction model and aspects of contextual dataset indicating whether the corresponding patients experienced flares may provide a labeled, expected output. Using the flare dataset and the contextual dataset, the prediction model may be trained with a supervised training approach. For instance, each training iteration may generate an output, which may be compared against the expected output, and backpropagation techniques may be used to refine the prediction model such that the prediction model generates an output closer to the expected output. Some non-limiting examples of the prediction model may include ensemble learners such as random forest, Light Gradient Boosting Machine (LGBM), XGBoost; and/or artificial neural networks such as Recurrent Neural Networks (RNN), Long Short-Term Memory (LSTM), Convolutional Neural Network (CNN), etc. It should however be understood that these are just examples and other prediction/statistical model should be considered within the scope of this disclosure.

This approach of training the prediction model is just an example and other approaches should also be considered within the scope of this disclosure. For example, the prediction model may be trained using an unsupervised training approach. In other example, the prediction model may be a statistical model, and step 806 may establish the statistical model using the retrieved datasets. Therefore, any type of data analytics to generate a prediction model or to establish a statistical model should be considered within the scope of this disclosure.

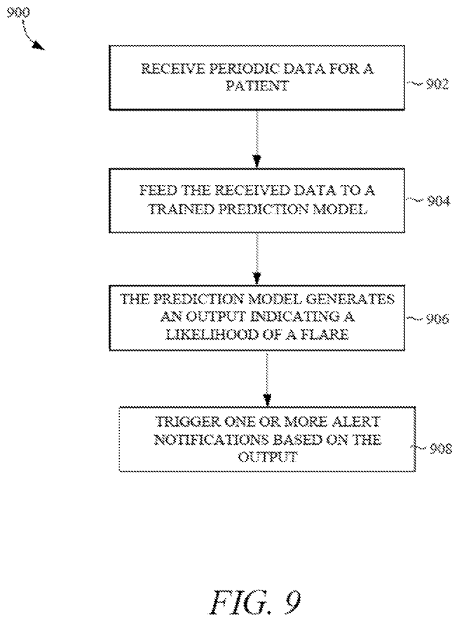

FIG. 9 shows a flow diagram of an illustrative method 900 of using a trained prediction model for flare prediction, according to some embodiments of this disclosure. The illustrative method 900 may be implemented by one or more computing devices (e.g., computing device 600 as used in the operating environment 500). It should be understood that the steps of the method 900 shown in FIG. 9 and described herein are merely illustrative and methods with additional, alternative, or a fewer number of steps should be considered within the scope of this disclosure.

At step 902 periodic data for a patient (e.g., a patient with atopic dermatitis or psoriasis) may be received. The periodic data may include scratch data for the patient for a time period. For example, the scratch data may be generated using passively collected movement data from a wearable and actively collected data from a healthcare application. The scratch data may indicate the number of scratch events, the severity of scratch events, and any other attributes associated with the scratch events. In addition to the scratch data, other contextual data may also be included in the periodic data. The other contextual data may include, for example, weather data, demographic data, etc. Any form of periodic data associated with the skin diseases should be considered within the scope of this disclosure.

At step 904, the received periodic date may be fed into a trained prediction model. The predication model may have been trained using the steps of the method 800. The trained prediction model should be understood also to include any type of established statistical model. Some non-limiting examples of the prediction model may include ensemble learners such as random forest, Light Gradient Boosting Machine (LGBM), XGBoost; and/or artificial neural networks such as Recurrent Neural Networks (RNN), Long Short-Term Memory (LSTM), Convolutional Neural Network (CNN), etc. In case of a statistical model, step 904 may include comparing a statistic (e.g., a z-statistic) to determine if the received periodic data generates is significantly closer a likely outcome (e.g., an outcome indicating a flare). It should however be understood that these are just examples and other prediction/statistical model should be considered within the scope of this disclosure.

At step 906, the prediction model (or a statistical model) generates an output indicating a likelihood of a flare. The likelihood of the flare may indicate corresponding of probabilities of the fed inputs being associated with a flare and not being associated with a flare. For example, the output may be probability of a flare 90% and probability of no-flare 10%.

At step 908, one or more alert notifications may be triggered based on the output of the prediction model. For example, if the prediction model generates a higher likelihood of a flare, an alert notification may be triggered to a healthcare application installed in a patient user device. The alert notification may indicate that a flare may be imminent, and the patient should reach out to a clinician. The alert notification may also provide options for the patient to initiate a synchronous or an asynchronous communication with the clinician. Another alert notification may be sent to the clinician's dashboard. This alert notification may identify the patient and indicate to the clinician that a flare may be imminent. The alert notification may also provide an option for the clinician to initiate a synchronous or an asynchronous communication with the patient, transmit a prescription to a pharmacy, and/or take any other mitigatory action.

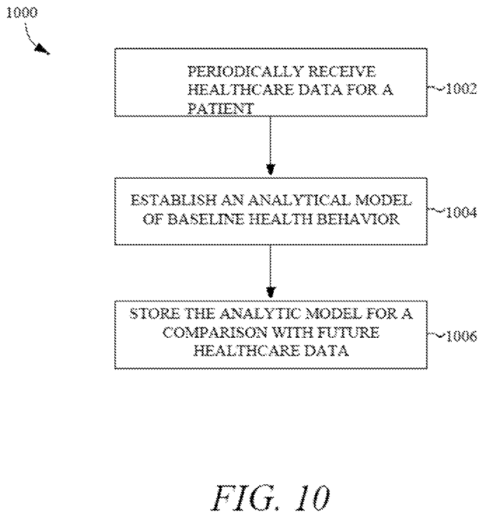

FIG. 10 shows a flow diagram of an illustrative method 1000 of generating an analytical model of a baseline disease behavior of a skin disease, according to the several embodiments of this disclosure. The illustrative method 1000 may be implemented by one or more computing devices (e.g., computing device 600 as used in the operating environment 500). It should be understood that the steps of the method 1000 shown in FIG. 10 and described herein are merely illustrative and methods with additional, alternative, or a fewer number of steps should be considered within the scope of this disclosure.

At step 1002 healthcare data from a patient (e.g., a patient with atopic dermatitis or psoriasis) may be periodically received. For example, the periodically received data may include healthcare data passively collected data from a wearable device (e.g., a smartwatch) and/or invisibles, and/or actively collected through a healthcare application (e.g., installed on a smartphone). The passively collected data may include, for example, movement data and other biological data (e.g., heart rate). The actively collected data may include, for example, the state of the patient's feeling, the prescription medication being taken, and/or other biological data entered by the patient.

This periodic collection of data may therefore be a longitudinal dataset indicating the progress of the skin disease (e.g., atopic dermatitis, psoriasis). Accordingly, this collection may be used at step 1004 to establish an analytical model of the baseline health behavior (and/or train machine learning model). The analytical model of the baseline health behavior may indicate, for example, a normal distribution of a combination of variables such as scratch events, the patient's state of feelings (e.g., stress and anxiety levels), patient's activity level, and/or any other attribute associated with the ongoing condition of the skin diseases. Some non-limiting examples of the machine learning model may include ensemble learners such as random forest, Light Gradient Boosting Machine (LGBM), XGBoost; and/or artificial neural networks such as Recurrent Neural Networks (RNN), Long Short-Term Memory (LSTM), Convolutional Neural Network (CNN), etc. It should however be understood that these are just examples and other prediction/statistical model should be considered within the scope of this disclosure.

At step 1006, the generated analytical model may be stored for comparison with future healthcare data. As the healthcare data is collected on an ongoing basis, such comparison may allow a determination whether the future collected healthcare data is within an expected distribution range or deviates significantly from the expected distribution range.

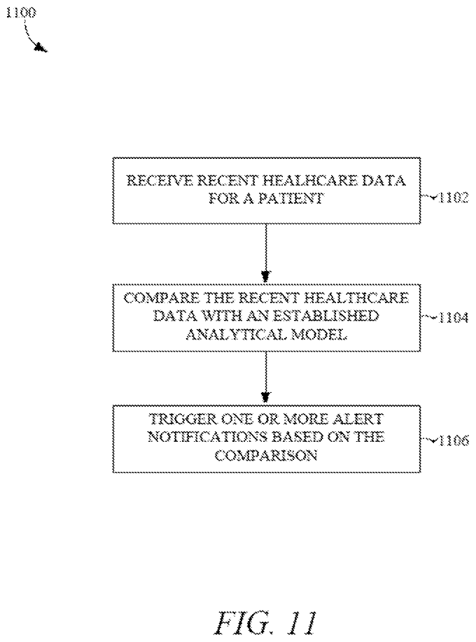

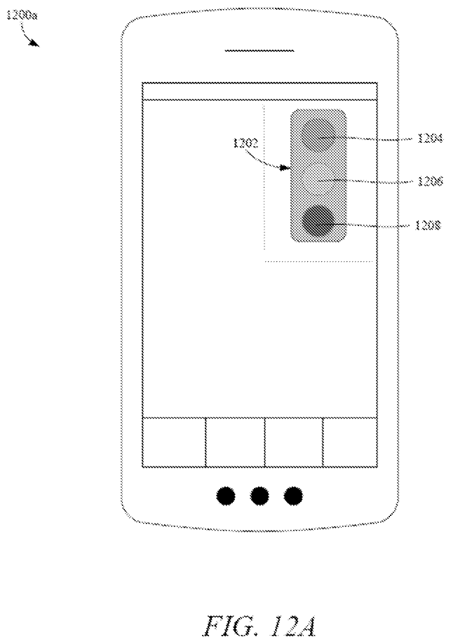

FIG. 11 shows a flow diagram of an illustrative method 1100 of deploying the analytical model to determine whether the current observed behavior deviates significantly from the baseline disease behavior, according to several embodiments of this disclosure. The illustrative method 1100 may be implemented by one or more computing devices (e.g., computing device 600 as used in the operating environment 500). It should be understood that the steps of the method 1100 shown in FIG. 11 and described herein are merely illustrative and methods with additional, alternative, or a fewer number of steps should be considered within the scope of this disclosure.

At step 1102, recent healthcare data for a patient (e.g., a patient with atopic dermatitis or psoriasis) may be received. The recent healthcare data may be collected passively, e.g., through wearables or invisibles, and/or actively, e.g., prompting the patient to enter data in a healthcare application. Such recent healthcare data may be gathered continuously, as the patient may be monitored in an ongoing basis.

At step 1104, the recent healthcare data may be compared with an established analytical model (e.g., analytical model generated by method 1000). The comparison may include determining whether the recent healthcare data shows a statistically significant deviation from an established baseline health behavior (as indicated by the analytical model). It should however be understood that the comparison with the established analytical model is just an example, and using a machine learning model for outcome prediction (e.g., predicting an aggravation of the disease) should also be considered within the scope of this disclosure. Some non-limiting examples of the machine learning model may include ensemble learners such as random forest, Light Gradient Boosting Machine (LGBM), XGBoost; and/or artificial neural networks such as Recurrent Neural Networks (RNN), Long Short-Term Memory (LSTM), Convolutional Neural Network (CNN), etc. It should however be understood that these are just examples and other prediction/statistical model should be considered within the scope of this disclosure.

At step 1106, one or more notifications may be triggered based on the comparison. For instance, if it is determined that the patient healthcare behavior has significantly deviated from the established healthcare behavior, an alert notification may be sent to the patient to initiate a synchronous or asynchronous communication with a clinician. Additionally or alternatively, another alert notification may be sent to a clinician to initiate a synchronous or asynchronous communication with the patient.

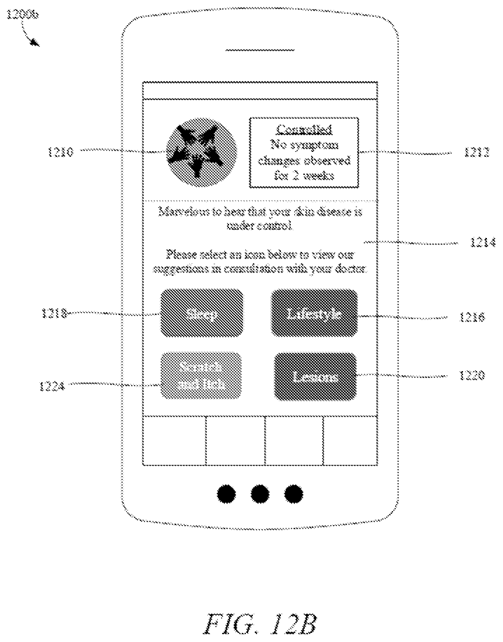

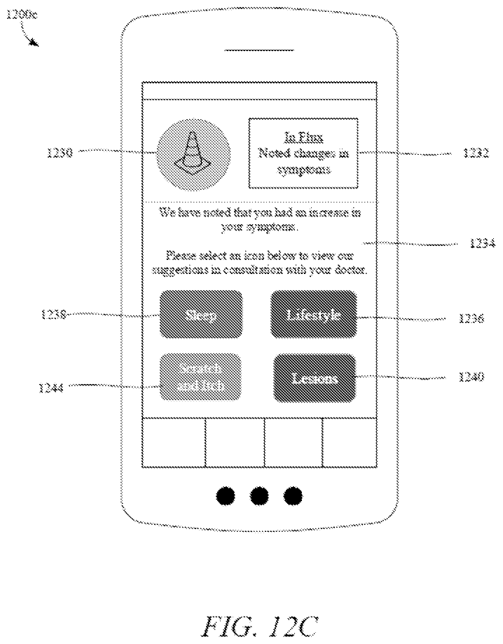

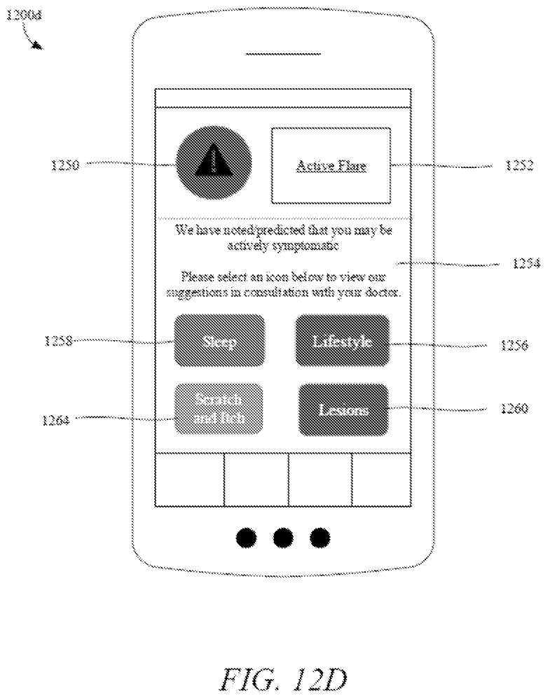

FIG. 12A-12D show an illustrative patient facing interfaces 1200a-1200d, according to several embodiments of this disclosure. The interfaces 1200a-1200d may be displayed at any type of patient facing device (e.g., patient user devices 502 as shown in FIG. 5). The interfaces 1200a-1200d may be generated based on analytics (e.g., machine learning/statistical analysis-based prediction) performed by any type of operating environment (e.g., operating environment 500 shown in FIG. 5, operating environment 700 shown in FIG. 7, etc.).

As shown in FIG. 12A, an initial interface 1200a may include a graphical object 1202. The graphical object may be based on a “traffic light”-type symbol for indicating alert or non-alert conditions. For example, a “green light” 1204 may indicate that the skin disease (e.g., atopic dermatitis, psoriasis, etc.) is in control and no changes have been observed in symptoms. A “yellow light” 1206 may indicate that the skin disease may be in flux. In other words, the yellow light 1206 may indicate that some changes in the symptoms have been observed. A “red light” 1208 may indicate that a prediction has been made for imminent flare and/or other type of worsening of the skin disease. A patient may select any of the lights 1204, 1206, and 1208 upon which one of updated interfaces 1200b-1200d may provide associated information, education, and/or action items.

FIG. 12B shows an illustrative updated patient facing interface 1200b, which may be generated when the patient selects the green light 1204 in the interface 1200a. In response, the updated interface 1200b may show an icon 1210 indicating the controlled skin disease and a text 1212 also indicating that the skin disease is under control. As shown the text 1212 may indicate that “no symptoms changes observed for 2 weeks.” However, this timing is merely used as an example and should not be considered limiting. The updated interface 1200b may further provide another text 1214 notifying the patient of the under-control skin disease. The text 1214 may also prompt the patient to click one or more of the appropriate icons 1218 (to receive suggestions or more information on sleep), 1216 (to receive suggestions or more information on lifestyle), 1224 (to receive suggestions or more information on scratch and itch), and 1220 (to receive suggestions or more information on lesions). These icons 1216, 1218, 1220, and 1224 may provide the corresponding suggestion or more information in consultation with the patient's clinician. For example, upon selecting the sleep icon 1218, the patient may receive suggestion on amount of recommended sleep to keep the skin disease under control.

FIG. 12C shows an illustrative updated patient facing interface 1200c, which may be generated when the patient selects the yellow light 1206 in the interface 1200a. In response, the updated interface 1200c may show an icon 1230 indicating the “in flux” skin disease and a text 1232 also indicating that the skin disease is in flux. As shown the text 1232 may indicate that “noted changes in symptoms.” The updated interface 1200c may further provide another text 1234 notifying the patient that the skin disease has had a recent increase in the symptoms. The text 1234 may also prompt the patient to click one or more of the appropriate icons 1238 (to receive suggestions or more information on sleep), 1236 (to receive suggestions or more information on lifestyle), 1244 (to receive suggestions or more information on scratch and itch), and 1240 (to receive suggestions or more information on lesions). These icons 1236, 1238, 1240, and 1244 may provide the corresponding suggestions or more information in consultation with the patient's clinician. For example, upon selecting the sleep icon 1238, the patient may receive suggestion on amount of recommended sleep to mitigate the recently noted symptoms.

FIG. 12D shows an illustrative updated patient facing interface 1200d, which may be generated when the patient selects the red light 1208 in the interface 1200a. In response, the updated interface 1200d may show an icon 1250 indicating an active flare skin condition and a text 1252 also indicating that an active flare has been observed or predicted. The updated interface 1200d may further provide another text 1254 notifying the patient an active onset of symptoms (e.g., flare) has been observed or predicted. The text 1254 may also prompt the patient to click one or more of the appropriate icons 1258 (to receive suggestions or more information on sleep), 1256 (to receive suggestions or more information on lifestyle), 1264 (to receive suggestions or more information on scratch and itch), and 1260 (to receive suggestions or more information on lesions). These icons 1256, 1258, 1260, and 1264 may provide the corresponding suggestions or more information in consultation with the patient's clinician. For example, upon selecting the sleep icon 1258, the patient may receive suggestion on amount of recommended sleep manage the active symptoms.

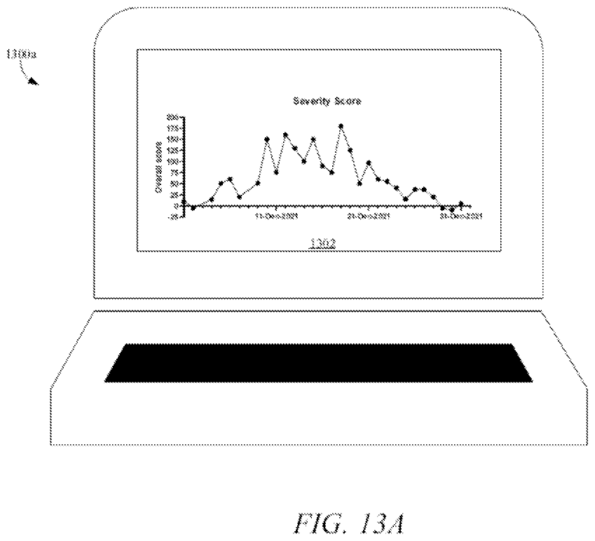

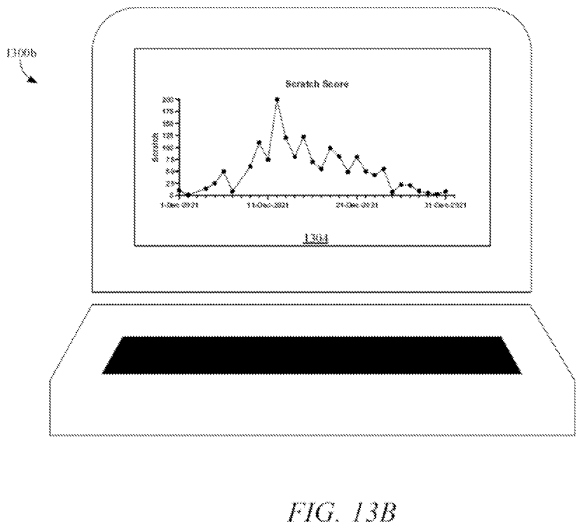

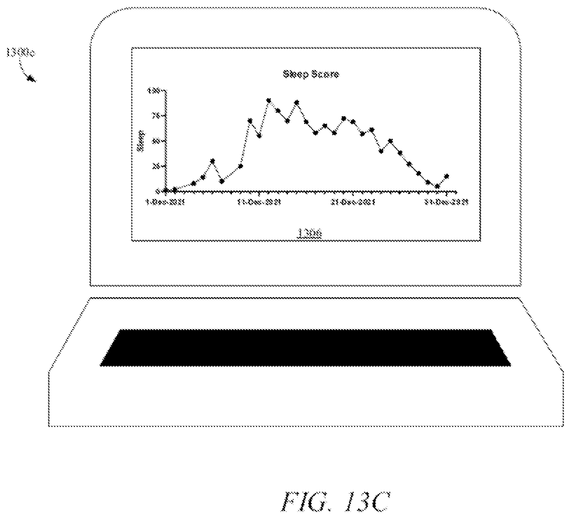

FIGS. 13A-13C show clinician facing interfaces 1300a-1300c, according to several embodiments of this disclosure. The interfaces 1300a-1300c may be displayed at any type of clinician facing device (e.g., clinician user device 508 as shown in FIG. 5). The interfaces 1300a-1300c may be generated based on analytics (e.g., machine learning/statistical analysis-based prediction) performed by any type of operating environment (e.g., operating environment 500 shown in FIG. 5, operating environment 700 shown in FIG. 7, etc.).

As shown, an initial interface 1300a may show a chart 1302 of a severity scores for any type of skin disease for a patient. The severity scores may be for a duration of time (e.g., a month). The interface 1300a may allow a clinician to observe the components of the severity scores. For example, a selection within the chart 1302 may generate an updated interface 1300b, as shown in FIG. 13B, that may show a chart 1304 of scratch scores forming the severity scores shown in the chart 1302. As another example, another selection within the chart 1302 may generate another updated interface 1300c, as shown in FIG. 13C, that may show a chart 1306 of sleep scores forming the severity scores shown in the chart 1302. Therefore, using the interfaces 1300a-1300c, a clinician may be able track the progress of a patient's skin disease.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

1. A computer-implemented method comprising:
retrieving, by a server, a scratch dataset, the scratch dataset comprising data records of corresponding scratch events of a patient population with a skin disease;
retrieving, by the server, a contextual dataset comprising data records of additional information associated with the corresponding scratch events;
training, by the server, a prediction model using a supervised training approach based on the scratch dataset and the contextual dataset;
receiving, by the server, periodic data indicating occurrences of scratch events for a time period of a particular patient with the skin disease;
feeding, by the server, the received periodic data into the trained prediction model; and
in response to the prediction model outputting a likelihood of a flare, transmitting, by the server, an alert notification to a user device of the particular patient.

2. The computer-implemented method of claim 1, further comprising:
in response to the prediction model outputting a likelihood of a flare, transmitting, by the server, a second alert notification to a clinician dashboard.

3. The computer-implemented method of claim 2, further comprising:
receiving, by the server, a patient communication message provided at the clinician dashboard in response to the second alert notification; and
transmitting, by the server, the patient communication message to the user device of the particular patient.

4. The computer-implemented method of claim 3, wherein the patient communication message comprises an indication for the particular patient to communicate with a clinician.

5. The computer-implemented method of claim 3, wherein the patient communication message is for a prescription medication to control the flare.

6. The computer-implemented method of claim 3, wherein the periodic data is received from a healthcare application running on the user device.

7. The computer-implemented method of claim 6, wherein the patient communication message is transmitted by the server to the healthcare application running on the user device.

8. The method of claim 1, wherein the data records of the corresponding scratch events comprise the frequency of the scratch events.

9. The method of claim 1, wherein the data records of the corresponding scratch events comprise the severity of the scratch events.

10. The method of claim 1, wherein the data records of the additional information comprise whether a corresponding scratch event was associated with a flare.

11. The method of claim 1, wherein the data records of the additional information comprise an association of a corresponding scratch event with at least one of weather, food intake, other infections, allergens, fabrics worn, presence of saliva at the skin disease site, dry skin, sweat level, stress level, exercise level, or hormonal level.

12. A computer-implemented method comprising:
periodically receiving, by a server, healthcare data from a healthcare application installed on a user device of a patient with a skin disease for a predetermined time period;
establishing, by the server, a baseline health behavior based on the healthcare data for the predetermined time period;
receiving, by the server, new healthcare data from the healthcare application installed on the user device;
determining, by the server, whether the new healthcare data has a significant deviation from the baseline health behavior; and
in response to the server determining a significant deviation from the baseline health behavior, triggering an alert notification to a clinician dashboard.

13. The method of claim 12, wherein the baseline health behavior is established by training a machine learning model.

14. The method of claim 13, wherein determining whether the new healthcare data has a significant deviation from the baseline health behavior comprises feeding the new healthcare data into the trained machine learning model.

15. The method of claim 12, wherein the healthcare data comprises at least one of passively collected data from a wearable device worn by the patient or actively collected data from the healthcare application installed in the user device of the patient.

16. The method of claim 12, further comprising:
in response to the server determining the significant deviation from the baseline health behavior, triggering a second alert notification to the healthcare application installed in the user device of the patient.

17. The method of claim 12, further comprising:
in response to the server determining the significant deviation from the baseline health behavior, facilitating, by the server, a communication between the patient and a clinician, via the healthcare application and the clinician dashboard, respectively.

18. A computer-implemented method comprising:
continuously receiving, by a computing device, healthcare data of a patient having a skin disease, from a wearable computing device worn by the patient;
periodically prompting, by the computing device, the patient to enter additional healthcare data in an interface generated by a healthcare application installed in the computing device;
transmitting, by the computing device to a remote server, the healthcare data and additional healthcare data;
receiving, by the computing device from the remote server, an alert notification in response to the remote server determining a likelihood of worsening of the skin disease; and
in response to receiving the alert notification, generating, by the computing device, a push notification indicating an action for the patient.

19. The computer-implemented method of claim 18, wherein the healthcare data received from the wearable computing device comprises movement data.

20. The computer-implemented method of claim 18, wherein the action for the patient includes at least one of filling a prescription, refilling a prescription, or communicating with a clinician.
